(19) United States
(12) Patent Application Publication
(10) Pub. No.: US 2015/0204834 A1
(43) Pub. Date: Jul. 23, 2015

(54) THERMODYNAMIC EQUIVALENCE SURROGATE TEST (TEST) FOR BIOEQUIVALENCE

(71) Applicant: Sarfaraz K. Niazi, Deerfield, IL (US)

(72) Inventor: Sarfaraz K. Niazi, Deerfield, IL (US)

(21) Appl. No.: 14/160,837

(22) Filed: Jan. 22, 2014

(51) Int. Cl. G01N 33/15

(52) U.S. Cl. CPC G01N 33/15

(57) ABSTRACT

A method for establishing bioequivalence of drug products by comparing the thermodynamic potential of the release of a drug substance from drug products according to a set of statistically differentiated dissolution profiles of an active drug substance contained in the drug product.

### BACKGROUND

Bioequivalence is defined in US 21 CFR 320.1 as “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study”. This definition has been emulated globally by every regulatory agency worldwide resulting in a creation of protocols and experimental designs requiring pharmacokinetic testing in humans. One assumption involved in all of these models is that since in most instances, the site of action may not be accessible for sampling, the indirect testing through study of the pharmacokinetic profiles is allows simulation of the profile of drug concentration at the site of action.

Establishing bioequivalence between two products is required in several situations:
Prototype formulations during early development and pivotal clinical trial formulations.
Mapping a process that relates Critical Manufacturing Variables (CMV), including formulation, processes, and equipment variables that can significantly affect drug release from the product.
Innovator formulations that differ from their NDA formulations as a result of scale-up and post-approval changes (SUPAC changes).
Implementation of improved manufacturing technologies.
Scale-up, changes in manufacturing locations, etc.
Multisource products filed for approval under a NDA as allowed under the Hatch-Waxman law.

The current guidelines proposed by global regulatory agencies require comparison of the pharmacokinetic profiles of the test and the reference drug product with specific ranges of acceptability of data to declare bioequivalence. The classical conventional human pharmacokinetic in vivo bioequivalence study employs a single dose, two period, two treatment, two sequence, open label, randomized crossover design comparing equal doses of the test and reference products in fasted, adult, healthy volunteers (e.g. n=24). These studies are an indirect measure of bioequivalence and supposed to represent a study of the drug concentration profile at the site of action.

The US 21 CFR 320.25(a) further codifies the universal belief that “No unnecessary human testing should be performed” and goes on to suggest: “The basic principle in an in vivo bioavailability study is that no unnecessary human research should be done.” The nature of exposure to healthy humans in conducting bioequivalence testing is exacerbated when highly toxic drugs or drugs requiring multiple dosing are administered. In an effort to reduce unnecessary exposure to humans, the regulatory agencies have made biowaivers (except in Japan) available for certain class of drugs that are not likely to have problem in achieving bioequivalence. This is provided under a biopharmaceutical classification system (BCS) wherein some classes of drugs are exempted from bioequivalence testing.

The in vitro bioequivalence testing has a long history of use. The US 21 CFR 320.33 has provided criteria to assess actual or potential bioequivalence problems. In the latter 1970s, drug products that had met these criteria were deemed not “bioproblem” drug products. In vitro studies were expected to correctly assess bioequivalence for products that were not “bioproblem” drug products. For instant or immediate release (IR) products not containing a “bioproblem” drug, FDA allowed DESI-effective drugs to be assessed for bioequivalence through in vitro studies alone. Since 1979, such products that passed bioequivalence testing were assigned an AA rating in FDA's “Approved Drug Products with Therapeutic Equivalence Ratings”. The US 21 CFR 320.24 also describes situations when in vitro studies can be used alone to document bioequivalence. Fact that the US FDA allows developers to challenge the bioequivalence testing opens the doors to more creative applications of the in vitro tests.

The use of dissolution testing in place of pharmacokinetic studies is generally allowed for those drugs that dissolve rapidly and where permeability of drug molecules across biological barriers in not problematic. This includes, for example, the drugs that dissolve at least 85% in 15 min (very rapidly dissolving) or in 30 min (rapidly dissolving) or less in pH 1.2, 4.5 and 6.8 dissolution media. These are the drugs where bioequivalence is self-evident making bioequivalence studies redundant.

There is however no global concurrence on a biopharmaceutical classification system for biowaivers; developers are not generally able to make global filings for bioequivalence testing for their products. There is a dire need to re-evaluate the scientific rationale behind the current methods of testing bioequivalence with aim to reduce or eliminate this testing where possible. The existing methodology used globally to demonstrate bioequivalence has many pitfalls that need to be reviewed prior to suggesting an alternate method of bioequivalence testing.

### SUMMARY OF THE INVENTION

One embodiment of the invention is method for measuring the thermodynamic potential of a drug product comprising comparing a test drug product with a reference drug product by a set of statistically differentiated dissolution profiles of an active drug substance contained in the drug product.

A second embodiment is a method for determining drug bioequivalence comprising measuring the thermodynamic potential between a test drug substance and a reference drug substance under conditions that yield a difference in their dissolution profiles, wherein equivalence of thermodynamic potential demonstrates drug bioequivalence. The matrix of dissolution data and comparability of the data between the test drug substance and the reference drug substance demonstrate drug bioequivalence.

A third embodiment is a method for determining drug bioequivalence comprising measuring the thermodynamic potential between a test drug substance and a reference drug substance under conditions wherein the inter-particulate binding and the intermolecular interactions are disrupted.

A fourth embodiment is a method for determining drug bioequivalence comprising measuring the thermodynamic potential between a test drug substance and a reference drug substance under thermodynamic stress conditions that modify the release characteristics of the test and reference drug substances to yield a difference in their dissolution profiles, wherein the stress condition is chosen based on its ability to alter the dissolution profile allowing the determination of drug equivalence. These stress conditions are, but not limited to, temperature, pH, dielectric constant, polarity (and bipolarity), osmolality, electrical field and various permutations and combinations of these conditions.

Another embodiment of this method comprises at least three conditions for each stressor that demonstrate a clear shift in the dissolution profile to generate the dissolution matrix applying the same conditions of testing to both the reference and test drug substances, and wherein a statistical analysis of the reproducibility of effects between the test and reference drug product establish drug bioequivalence.

Another embodiment comprises two stressors and three conditions applied to both the reference and test drug substances to yield a matrix of nine test profiles.

Another embodiment involves a set of dissolution profiles obtained in a variety of dissolution media. The dissolution medium pH can range from 1-14. The dissolution medium has an osmolality can range from 100 to 1000 mOsm/L. The dissolution medium may have a dielectric constant value of more than 1 and/or less than 81. The dissolution medium may further comprise a non-ionic surfactant, an ionic surfactant or a combination thereof. This surfactant may be below its critical micelle concentration.

In another embodiment, the dissolution medium may comprise a mixture of a polar liquid, a non-polar liquid or a plurality of polar and non-polar liquids. The chemical composition of the dissolution medium may comprise an oil-in-water or water-in-oil emulsion. The chemical composition of the dissolution medium may comprise a coacervate or milk.

In another embodiment, the dissolution media may be subjected to a variety of physical conditions, including but not limited to, temperature within a range of 5 to 50° C.; an electrical field applied to the dissolution medium from 1-50 volt direct current; mixing of the dissolution by a stirrer, paddle, mechanical vibrator, or ultrasonic vibrator placed inside the dissolution medium; or physical agitation such as rocking, shaking, rotating and vibrating the dissolution medium.

The dissolution profiles may be created for less than one hour. Or the dissolution profile may be created under sink conditions or non-sink conditions.

The drug product may be in any form, such as a solid, semi-solid, liquid, powder or a gel.

### DETAILED DESCRIPTION OF THE INVENTION

### Site of Action Requirement

The official definition of bioequivalence as enumerated above requires demonstration of equivalence of concentration at the site of action. The fact that the site of action of most drugs is not known and not possible to sample makes this universal definition questionable. When a new chemical entity (NCE) is developed, the developer is required to provide data on the pharmacokinetics (what body does to the drug) profile as well as the pharmacodynamic (what the drug does to the body) profile to establish safe dosing. The developer is not required to identify the site of action, nor the mechanism of action and the dosing is never based on any evaluation of the concentration at the site of action. The bioequivalence testing under conditions enumerated above is conducted to assure that the two products are equally effective and thus interchangeable. Requiring an assessment at the site of action enhances the requirements beyond what is required for the NCE despite the established demonstration of dosing and safety of the active ingredient. The use of pharmacokinetic profiles to simulate the concentration at the site of action is also flawed since it assumes a linear dose response making the pharmacokinetic profile a poor choice to meet the basic requirement of bioequivalence.

In brief, the official definition of bioequivalence requiring comparisons between two products to evaluate any difference between them based on the concentration achieved at the site of action is at best impractical and at worse irrelevant to the goal of demonstrating bioequivalence. There is a need to revise the definition of bioequivalence. One choice will be to require statistically insignificant pharmacokinetic profiles but that too will be unacceptable as an indirect test of the difference in the drug products; the purpose of bioequivalence testing is to decipher any differences in the two drug products, not the safety and efficacy that has already been established.

### Statistical Modeling Errors

The bioequivalence evaluation based on pharmacokinetic profiling is achieved through use of complex statistical models to account for the high variability (inter-subject, intra-subject) of the data collected. The studies are powered to various levels of confidence intervals and the number of subjects enrolled depends on the coefficient of variation in the data collected. As a result, many developers conduct a pilot study to establish this variability to correctly power their final study. Despite years of developing suitable statistical models, errors remain evident in the bioequivalence testing making these studies less useful.

Two types of errors are common in bioequivalence studies: type I and type II (Table 1)

TABLE 1
Types of errors in hypothesis testing

| Result of Testing | Ho is true | Ho is false |
|---|---|---|
| Fail to reject Ho | Correct decision | Type II (producer risk, β) |
| Reject Ho | Type I (consumer risk, α) | Correct decision |

Ho: Products are not bioequivalent

In bioequivalence testing, the null hypothesis states that products are not bioequivalent (Ho), while the alternate hypothesis states that products are bioequivalent (H1). Type I error occurs when products are erroneously concluded to be bioequivalent when they are not bioequivalent. Type I error represents a risk to the consumer (i.e., a health risk to the patient). Type II error occurs when products are erroneously concluded to be not bioequivalent when they are bioequivalent. Type II error represents a risk to the producer as a good product is rejected.

Assuming that the conventional human pharmacokinetic in vivo bioequivalence testing is a perfect indication of whether products are bioequivalent, the extent that products pass Class I (BCS) with rapid dissolution but fail in vivo bioequivalence testing is analogous to the Type I error rate of in vitro testing.

Highly variable drugs (HVDs) are drugs with high within-subject variability (ANOVA-CV≧30%) in Cmax and/or AUC. HVDs typically have flat dose response curves and large therapeutic windows, such that clinically important adverse drug reactions (ADRs) occur at much higher doses than those required for efficacy. Currently in the USA, the same conventional bioequivalence statistical analysis [i.e., AUC and Cmax; log-transformed data; ANOVA model with period, sequence, subject (seq), and treatment; and 90% confidence intervals must fit between 80-125%] is applied to HVDs, as well as non-HVDs. It is well appreciated that HVDs often require a greater numbers of subjects than non-HVDs, in order to avoid type II errors when products are erroneously concluded to be not bioequivalence when they are in fact bioequivalent. High variability is a frequent basis for low in vivo bioequivalence study power, necessitating larger number of subject to achieve sufficient power. Drug formulations associated with an intra-individual variability of 35% or more generally fail to meet bioequivalence criteria at an astronomic rate of over 85%. In spite of this pattern of high in vivo bioequivalence testing failure for HVDs, evidence indicates that high variability is frequently not due to poor product quality; an aspect that is primarily investigated in bioequivalence testing, but it is mostly confounded within the biological variability.

In addition to being subjected to type I and type II errors, the conventional in vivo bioequivalence testing also is imperfectly designed. In traditional bioequivalence testing, the residual variance is composed of (a) analytical variability, (b) within-subject variability in ADME, (c) within-formulation variability, (d) subject-by-formulation interaction, and (e) unexplained variability. The conventional two period designs cannot separate these variance components. Hence, passing the traditional bioequivalence test assumes that the two products have sufficient product quality in that within-formulation variability and subject-by-formulation interaction are small. Traditional bioequivalence testing does not consider differences in within-subject variability between test and reference. Replicate designs where each product is administered twice allows partitioning of the subject-by-formulation interaction from residual variance and estimation of within-subject variability of each the test and reference. Conventional in vivo bioequivalence testing is not sensitive to detecting a subject-by-formulation interaction effect or a reference that is a highly variable drug product (HVDP), except of course that such increase in variability will necessitate an increase in subject numbers to establish bioequivalence.

While a few theoretical scenarios provide a basis for a subject-by-formulation interaction effect, it perhaps is surprising that conventional in vivo bioequivalence testing is not sensitive to detecting a reference that is a HVDP. The term HVDP differs from HVD. HVDs are drugs with high within-subject variability (ANOVA-CV≧30%) in Cmax and/or AUC. HVDs are typically associated with high first pass. A HVDP is a formulation of poor pharmaceutical quality where the drug itself is not highly variable, and where within-formulation variability [e.g. capsule to capsule variability] is large. Conventional in vivo bioequivalence testing is not sensitive to detecting a reference that is a HVDP.

The weakest reason to favor in vivo bioequivalence over in vitro testing is that the two approaches can provide different results. When products are truly bioequivalence (or truly not bioequivalent), both tests can be correct, both incorrect, or one correct and the other incorrect. Lack of concordance between in vitro and in vivo results reflects type I and type II errors of each approach. This does not reduce the importance of in vitro testing, only the demonstration of the lack of correlation.

Situations where in vitro test should be viewed as preferred include Class I drugs with rapid dissolution, Class III drugs with very rapid dissolution, and HVDs with rapid dissolution and that are not bio (equivalence) problem drugs. These situations represent a substantial majority of drugs. Class I and III drug make up about 50% of all marketed oral solid drug products. Upwards of 31% of drugs are HVDs. Since most HVDs show high first pass metabolism and since many such drugs may be expected to be highly permeable, it can be estimated that a substantial majority of drugs are candidates for in vitro bioequivalence testing as the preferred bioequivalence test.

The current method of statistical modeling of pharmacokinetic studies to elucidate difference in the quality of drug products is inappropriate and wasteful.

### Waiver System

The Biopharmaceutic Classification System (BCS) classifies active substances into four different groups (Table 2) according to their aqueous solubility at the highest dose in a volume of 250 ml and intestinal permeability. This system of classification was proposed by Gordon Amidon in the mid-1990s. The idea was that when a drug formulated as an immediate release solid oral drug product has an in vivo high solubility into the gastrointestinal tract and has a high permeability (class 1 drugs) the rate and extent of drug absorption is expected to be equivalent to the in vitro dissolution test, being 85% dissolved in less than 30 min with three different buffers, making in vivo bioequivalence demonstration unnecessary. This approach has been accepted by the FDA and EMA but is still rarely used, at least in Europe. Nevertheless the EMA has gone one step further, and has released new bioequivalence guidelines that include recommendations on BCS-based biowaivers. Biowaivers would be applicable to products containing BCS class 1 drugs which exhibit high solubility, complete absorption and rapid in vitro dissolution and also to pharmaceutical products containing substances exhibiting high solubility, limited absorption (BCS class 3) and very rapid in vitro dis-solution, once the product has been previously justified for the biowaiver and has demonstrated other specific requirements concerning active substances and excipients. Also the new EMA guideline said that ‘excipients that might affect bioavailability are qualitatively and quantitatively the same. In general, the use of the same excipients in similar amounts is preferred’. More than that, the WHO had relaxed the solubility ratio and permeability criterion for class 1 and class 3 allowing too some bioexceptions in pharmaceutical products containing BCS class 2 drugs that are weak acids with dose solubility ratio of 250 ml or lower at 37° C. over a pH range of 1.2-6.8. Such waivers would increase the speed and decrease the cost of bringing orally administered multisource therapeutics to market.

Recent literature analyzing the bioequivalence outcomes and their BCS classification has shown that the basic premise of awarding waivers based on solubility and permeability does not hold. It has taken decades to accumulate enough data to prove the uselessness of the BCS waivers. Comparisons between pharmaceutical products with active substances from the four BCS classes do not show any differential characteristics of each class in terms of n, inter and intra-subject variability for Cmax or AUC. Despite the usually employed test dissolution methodology proposed as quality control, pharmaceutical products with active substances from the four classes of BCS showed non-bioequivalent studies.

Table 2 also shows the relative prevalence of the various classes of drugs in the BCS. It shows that more than 50% (Class I and III) of drugs fall in the high solubility range where the testing may not be required. This means that most drugs currently waived for bioequivalence testing may not be meeting the quality requirements.

The most common criteria for classifying these drugs, in vitro-in vivo correlation (IVIVC), has little value because the dissolution profiles (the in vitro profile) and blood levels (in vivo profile) can be totally independent of each other. There has been too much emphasis given to IVIVC in the past without realizing that combining a biological process with a chemical process defeats the purpose of the exercise.

TABLE 2
Biopharmaceutic Classification System and correlation
with in vitro- in vivo correlation (IVIVC) Distribution
of drugs in various BCS classes.

| Class | % Prevalence | Solubility | Permeability | Likelihood of IVIVC |
|---|---|---|---|---|
| I | 30-36%, | High | High | IVIVC expected |
| | | | | (if dissolution is |
| | | | | rate-limiting step) |
| II | 30-34% | Low | High | IVIVC expected |
| III | 19-28% | High | Low | Little or no IVIVC |
| IV | 3-7% | Low | Low | Little or no IVIVC |

IVIVC: in vitro-in vivo correlation

The basic premise in the BCS is based on two parameters of the chemical entity: solubility and permeability, both of which are inherent properties of the chemical entity and have nothing to do with differences in the drug products. These two parameters are also inter-dependent wherein permeability is a function of both aqueous and non-aqueous solubility. Using a thermodynamic property of the active ingredient to establish the quality of a drug product is deeply flawed. Drugs having different permeability potential behave differently and drugs that have little permeability issues are generally granted biowaivers. These biowaivers can be questioned as being a carte blanch to a certain class of drugs allowing variations in the drug products that may be relevant to the quality of the drug products.

### Biological Variability

For drugs that have problems in crossing the biological barriers, blood level studies are required assuming that somehow the release of drugs at the site of administration can be monitored through blood level monitoring. Reality is that in the case of drugs with problem in absorption, the blood levels are less likely to discriminate between the drug products since the variability introduced by the drug products is much smaller compared to the variability in blood levels resulting from the variations in the absorption properties of the drug.

Post-absorption events such as metabolism and enterohepatic recycling further add to the variability in the pharmacokinetic profiles. Recognition of this inherent variability has resulted in bioequivalence testing requirements that vary across various regulatory agencies to account for the confounding factors such as within-subject variability in absorption, distribution, metabolism and excretion (ADME), enterohepatic recirculation, etc. For example, the Canadian agency does not require a confidence interval for Cmax, but corrects for drug content; FDA requirements differ. The CPMP/EMEA guideline allows broadening the bioequivalence limits (e.g. 75-133%) under certain situations. There are also proposals to broaden the bioequivalence limits according to the within-subject variability of the reference. Additionally, in vivo bioequivalence testing is subject to metric issues, where Cmax is not viewed as an ideal metric for rate, such that early exposure may sometime need to be measured. These limitations of in vivo bioequivalence testing have been frequently discussed, resulting in a range of different criteria to assess bioequivalence from pharmacokinetic data.

The differences between two drug products are confounded by the biological variability related to drug absorption and disposition making the current method of testing bioequivalence irrelevant. This observation is clearly demonstrated in the range of pharmacokinetic data considered acceptable for bioequivalence demonstration. Most bioequivalence studies allow a window of acceptability that is mostly 80-125%, or allowing almost 50% variability for the products to be bioequivalent. It is easily understood that the variability in the delivery of drug to the site of administration is likely to be much smaller than the window of variability allowed when blood levels are used as a surrogate for the concentration of drug at the site of administration.

### Multiple Dosing

Besides the high variability of the pharmacokinetic profiles because of biological reasons, more complications in using in vivo bioequivalence testing result where multiple dosing is required. First, the drug levels achieved in multiple dosing confound the variations in each dose resulting in a pharmacokinetic profile less capable of differentiating between drug products; second, since the exposure to healthy subjects can be large, the studies are often conducted in patients, where the drug clearance mechanisms may be compromised. This also adds to the cost of the study substantially. In multiple dose studies, each subsequent dose also has the ability to non-linearly affect the pharmacokinetic profile resulting in extremely complex pharmacokinetic and pharmacodynamic profiles that may have little correlation with the quality of the drug product.

### Dissolution Testing

The current methodologies for dissolution testing as used in awarding biowaivers and to monitor the quality of drug products are based on a choice of dissolution medium that is supposed to emulate the site of drug administration. Recent studies have shown that this approach of dissolution testing to establish IVIVC to secure a biowaivers do not correlate with bioequivalence outcomes. Fact is that this milieu for the drug delivery systems is not well established and is also a highly variable as the drug product passes through a course of anatomy. The correct approach should be to study dissolution rates under not a single but a variety of conditions regardless of their relevance to physiologic conditions; once a variety of dissolution profiles are established under different conditions, a comparison between a reference and a test product should suffice declaration of bioequivalence. The rationale behind this proposition is that the two bonding forces within a drug product, inter-particulate and intermolecular, create the differences in the release rates and these can be studied only under a variety of conditions of dissolution. A comparable change in the dissolution profile induced by a variety of conditions will point to similarity of the inter-particular and intermolecular bonding and thus the quality of drug products.

There is no single universal dissolution media that a priori predicts in vivo drug dissolution. There is no single in vitro (and in situ) permeability test condition that mimics the complex intestinal mucosa that drug can experience over the course of its passage through the gastrointestinal lumen. To overcome this, multi-condition dissolution testing and multi-condition permeability testing involving mainly various pH levels has been recommended. All of these studies and suggestions continue to force an IVIVC that does not truly exist.

### Complex Systems

Whereas detailed guidelines are available for instant or immediate release drug products, complex systems where controlled or sustained release is intended or where blood levels are not a predictor of the response and toxicity, extremely complex bioequivalence tastings are mandated and in some cases this means conducting limited clinical trials. This defeats the basic purpose of simplifying the process of approval of generic products or to validate change controls. This complexity arises from the way bioequivalence is defined; if the two drug products have reproducible patterns of release and the underlying factors that produce these profiles are similar then the need for any complex testing will be obviated.

### One Time Testing

Bioequivalence testing is currently required to be conducted only once when the needs arise as specified above. This is the most serious flaw in the current requirements. In most instances, the sample tested might present an early change or development. The Phase I materials are generally modified when reaching full commercial production and these changes are likely missed out in the evaluation of bioequivalence. In the 1980s, the FDA got hit with several fraudulent filings of ANDAs where the developers used the reference drug products for the test substance as well; it took years to decipher this. This would not have been possible if there were a requirement of continuous comparison of the test and reference products. Minor process changes do not require bioequivalence re-testing but over period of time several minor changes may have brought significant changes that are currently overlooked.

### Time

One of the basic purposes of initiating waivers of clinical trials in ANDAs was to allow faster development of generic products. Whereas waiving clinical trials did reduce the time considerably, the remaining in vivo requirements in bioequivalence trials extend the product development time this can be significantly curtailed if the purpose of these studies is re-examined to conclude that none of these testing is required to assure a lifetime of quality of products.

### Cost

The cost of bioequivalence studies, particularly when fed and multiple dose studies are required, remains very high and keeps out of market many developers; additionally, most developers engage lower cost vendors where the quality of data may not be as dependable. Eliminating the requirements of bioequivalence testing will increase the number of multisource products and reduce the cost of these drugs to consumers.

### Interchangeability

The basis tenet of the Hatch-Waxman law was to provide a generic interchangeable alternate at a lower cost. The awarding interchangeability of products has the assumption that the reference and the test products continue to be similar over the lifetime of the product. Nothing can be farther from truth. Without continuous monitoring of the quality of drug products against the innovator or the earlier product (where it can be based on a pre-determined criteria), the lifetime quality of drug products cannot be guaranteed. Additionally, the innovators, against whose product is the interchangeability granted, may change its process of manufacturing, specification and sourcing of API, create a new dissolution profile, alter the formulation—none of which is confided with the generic drug product manufacturers and even when this is disclosed, the generic manufacturer is not required to adopt these changes. This creates a serious problem in keeping the two products interchangeable and may be a cause of the reported failures of the generic products. By requiring the generic manufacturers to continuously compare their products with the innovator product should obviate this problem. However, repeated bioequivalence testing is not justified and in vitro testing on a continuous basis comparing the innovator and the generic product is required to assure that the generic products remain interchangeable.

### Global Harmonization

Global regulatory agencies have different opinions on biowaivers. For example, no biowaivers are allowed in Japan. Other agencies use a variety of methods of classifying the drugs for biowaivers and the type of in vitro testing required; this makes global filing of generic products difficult and expensive task for developers. Even when there is a concordance on the protocol of in vivo bioequivalence testing among the agencies, the requirements for statistical treatment of data differs. These differences point out to the weaknesses inherent in the utilization of bioequivalence testing. A better global harmonization can be achieved by eliminating the need for bioequivalence testing and replacing it with appropriate in vitro tests that can be readily replicated.

### A New Approach to Bioequivalence Demonstration

Bioequivalence testing is supposed to identify any substantial differences in drug products, not to evaluate the clinical efficacy and toxicity. The chemical drug products used in drug products can be thoroughly analyzed and compared and the regulatory agencies must demand better comparison profiles of the chemical drug products including impurities, crystalline structure, solvates and many other properties. Once that is established, the products are declared chemically equivalent. Whether a developer chooses the same formulation of its generic product is neither necessary nor possible—this may well be a pharmaceutical alternate rather than pharmaceutical equivalent. From this point forward a comparison of the quality of two products is mainly governed by the efficacy of the drug delivery system.

If an identical molecule is released in an equal concentration and rate at the site of administration from two drug products, then these should be declared bioequivalent. To fully understand and exploit this suggestion, we need to examine and appreciate the course of a drug molecule from the drug product to the site of action as given below:
Intact drug delivery system [symbol] Dispersed drug delivery system [symbol] Drug substance in solution at the site of administration [symbol] Membrane transport [symbol] Drug substance in the biosystem (e.g., plasma) [symbol] Drug substance at the pharmacologic action site [symbol] Clinical or Toxic response

The steps in bold represent the processes that are independent of the quality of the drug product; these are also the processes that are highly variable and should not be made part of the evaluation of the drug product, whose only job is to deliver the active pharmaceutical ingredient at the site of action; when the concentration and the rate of delivery at the site of administration is identical, the drug products should be declared bioequivalent. However, determining the concentration and the rate of availability at the site of administration itself poses problems; unless we can provide a direct sampling, which is not possible, a surrogate test needs to be developed. A surrogate test need not be correlating the properties of the drug delivery system at the site of administration but assure that the two drug products will behave similarly at the site of administration. This concept of comparison is novel and does not replicate the same errors that were committed in developing the use of pharmacokinetic profiling to simulate the drug concentration at the site of action.

Fortunately, sound scientific principles can be applied to providing an assurance of the quality of drug products if the mechanism of delivery of active pharmaceutical ingredient at the site of administration of better understood. Generally, there are two physical and chemical forces, inter-particulate bonding and intra-molecular bonding that must be overcome to yield a monomolecular solution. The key to evaluating these bonding forces comes from dissolution profiles obtained under a variety of conditions that can change the ease with which these forces are overcome during dissolution. If the two products demonstrate a similar shift in dissolution rate, the assumption that the bonding forces are the same or can be overcome identically can be assumed.

Drug molecules reaching the site of action face a multitude of inevitable barriers—the biobarriers—the classical bilipoidal layers that are present in our body to protect us from the entry of foreign chemicals that can harm us. These barriers cascade throughout the body leading all the way to the cellular level of the site of pharmacologic response. The only function of a drug product is to deliver the active drug at the site of administration. From this point forward, the disposition of drug molecules is controlled by the body physiology independently of the drug product.

There is a need to fully exploit dissolution testing to correlate with the release characteristics at the site of administration rather than correlate it with the pharmacokinetic profile.

The new and novel method proposed here is to test the dissolution rate of the drug products under conditions that may have little to do with the physiological environment and more to do with the chemical potential of the drug products. The proposed testing differs from the current dissolution methods that are limited to a single condition, more particularly, a physiological condition that fails to differentiate between the drug products as they might behave at the site of administration.

### Thermodynamic Potential

Thermodynamic forces drive the factors that cause drug molecules to be released from a drug product: solubility, surface area, temperature, the strength of inter-particulate binding, inter-molecule binding forces, and a complex interdependence of these factors.

Chemical reactions proceed with the evolution or absorption of heat. This heat flow represents differences in chemical energy associated with the rearrangement of atoms in molecules, the making and breaking of bonds to form new substances. When measured at constant pressure this is the enthalpy change (ΔH) for the reaction.

Physical changes can also involve heat. Typically the dissolving of a solid in water will involve measurable heat. There is not absolute agreement on whether dissolving itself should be categorized as wholly physical, partly chemical, etc., but intermolecular forces are certainly involved (at the very least). Regardless of the appropriate “label” for dissolving as a process there is an overall energy term. It is known as the heat of solution, ΔHsoln.

It may not be possible to know the precise mechanism for a particular dissolution, but in a hypothetical generalized scenario there would be at least three energy changes involved:
solute particles are separated from the solid mass (energy is absorbed, ΔH1)
solvent particles move apart to make space for dissolved solute (energy is absorbed, ΔH2)
solute and solvent particles are attracted to one another (energy is released, ΔH3)

For most solids dissolving in water, the sum of the first two terms is greater than the third and thus dissolving is frequently endothermic (ΔHsoln=+) and solubility generally increases with increasing temperature. When heats of solution become very highly positive it is often because the solute and solvent are dissimilar and, in the extreme case, immiscible. The old rule of “like dissolves like” is an approximation, but an important one.

The force that determines the penetration of drug molecules across biological barriers is dependence on the chemical potential of the drug at the site of absorption; while concentration may be subject to several variants; the chemical potential is the correct measure of the effectiveness of a drug product. According to Scheme 1, the free energy of the system changes towards negative free energy, as the drug product transforms from a single drug product to a monomolecular solution. Based on the Gibbs-Helmholtz equation:

ΔG=ΔH−TΔS

The spontaneous conversion of a highly aggregated drug product into highly dispersed monomolecular solution is entropy driven that supersedes the effects of any enthalpy-driven factors leading to a negative free energy. Once a maximum dispersion of drug molecules is achieved, the chemical potential of the molecular drug products is maximized.

Dissolution profiles represent the thermodynamic transformation of drug products; this phenomenon can be used to test the differences between two drug products if an orthogonal testing approach is adopted where the purpose is to decipher the thermodynamic potential differences between the tested drug products.

A comprehensive approach to compare the thermodynamic potential of the two drug products requires a systematic approach:
1) Assure chemical equivalence. This means that the API has exactly the same chemical structure as the innovator or another product that is compared with. This includes the profiling of the impurities, chemical variants such as stereoisomers, chiral forms, crystalline structure, amorphous component, solvates and hydrates.
2) Assure pharmaceutical equivalence. This means the design of the drug delivery system, the physical characteristics of all components (e.g., particle size), including excipients. Also assured at this stage is the stability profile of the API in the compared drug delivery systems. Pharmaceutical equivalence is not required and alternates can be used.
3) Assure in vitro drug release equivalence. Dissolution profiles derived under conditions that are capable of capturing any differences in the ability of the drug product to release the drug are established. Currently, the US FDA database on dissolution testing conditions is limited to a set of specific conditions that do not provide adequate comparison opportunity. The new testing method involves a variety of dissolution conditions that show a definite shift in the dissolution profile; a similar shift in the dissolution profile under various thermodynamic stressors (dissolution conditions) establishes bioequivalence and a method for lifetime assurance of drug product quality.

This surrogate approach labeled as Thermodynamic Equivalence Surrogate Test (TEST) for Bioequivalence has significant advantages over the current methods of testing. This includes: (a) reduced costs to developer, (b) assessment of product performance more directly and routinely, (c) obviate exposure to human subjects on an ethical ground (d) provide a monitoring of drug product quality throughout the lifecycle of the product, and (e) universal application to all types of dosage forms, from immediate release to timed, programmed, sustained and long-term release.

### Thermodynamic Equivalence Surrogate Test (TEST™) for Bioequivalence

The thermodynamic potential (chemical potential) of a drug product is the key determinant of its effectiveness and a comparison of this potential between a test and a reference drug product should suffice to establish their bioequivalence. The strength of this testing depends on the robustness of the testing conditions developed as these will be highly dependent on the nature of the drug and the dosage form. As a result, the given approach can be equally applicable to all types of dosage forms including immediate, controlled, timed, sustained or any other type of release profile since the approach is independent of the functionality of the drug and its evaluation and the comparisons are limited to the ability of the drug product to demonstrate identical thermodynamic potential.

Thermodynamic equivalence can be readily established if the dissolution profiles of the test and reference drug products are compared under conditions that yield a variance in the dissolution profile; the premise being that if both reference and test drug products show a similar shift in the dissolution profile then the underlying thermodynamic potential will also be the same. Providing multiple profiles in a variety of conditions of dissolution will pick out even smaller yet relevant differences between the test and the reference drug products, not possible to be identified by the current methodology.

Currently, the most common dissolution testing is done at fixed conditions:
US FDA suggests 50 rpm (paddle) or 100 rpm (basket); 900 ml; USP buffer; 37° C.
WHO suggests 75 rpm (paddle) or 100 rpm (basket); 900 ml; USP buffer; 37° C.
EU: nothing explicit.

No surfactants and other additives are allowed. There is a need to substantially expand the scope of dissolution testing. In brief, conditions of dissolution that alter the thermodynamic stress during dissolution should be developed to create a matrix of dissolution data and the overall comparability of these data between the reference and the test drug product would establish thermodynamic equivalence.

The fundamental principle of creating thermodynamic stress involves a clear understanding of the types of forces that come into play in the dissolution of drugs. The first is the inter-particle binding in a solid drug product and the other is intermolecular interaction; both of these should be over come to allow a monomolecular dispersion of the active drug at the site of absorption.

The thermodynamic stress conditions that modify the release characteristics of the drug product may include temperature, pH, dielectric constant, polarity (and bipolarity), osmolality, electrical field and various permutations and combinations of these conditions. The selection of the stress condition should be based on the ability to alter the dissolution profile significantly for both the test and the reference drug product leading to a possibility of comparison and thus establishment of bioequivalence if the dissolution profiles are superimposable.

The dissolution profiles can be tested using such parameters as concentration at various time intervals, the partial and cumulative AUC under the concentration time curve and then comparing the dissolution profile of the test and reference drug product and prove comparability by superimposable dissolution profiles.

When comparing the test and reference products, the dissolution profiles are currently compared using a similarity factor (f₂). The similarity factor is a logarithmic reciprocal square root transformation of the sum of squared error and is a measurement of the similarity in the percent (%) of dissolution between the two curves.

f2=50+log {[1+(1/n)?t=1*n(Rt−Tt)2]−0.5*100}

Two dissolution profiles are considered similar when the f₂ value is ≧50. To allow the use of mean data, the coefficient of variation should not be more than 20% at the earlier time points (e.g., 10 minutes), and should not be more than 10% at other time points. Note that when both the test and the reference products dissolve 85% or more of the label amount of the drug in less 15 minutes using all three dissolution media recommended above, the profile comparison with an f₂ test is unnecessary.

The use of TEST requires developing a new method of declaring similarity where the drug product is not allowed to dissolve as fast so that the differences can be studied. The thermodynamic stressors listed below are more typical than most relevant; there can be a stressor highly specific to the drug of choice and in all instances a permutation and combination of these stressors can be studied to provide a matrix of tests that will allow an objective direct measure of the differences between the drug products and thus a comparison of their quality in delivering the active drug products at the site of absorption.

### Temperature

The temperature of 37° C. is most commonly used with intent to connect this condition to a physiological milieu; in TEST application, the objective is to identify the differences in two drug products in environment that causes a shift in the dissolution rates. Any relevance to physiological conditions can be ancillary but not of any other value. For thermodynamic stress purposes, the temperature needs to be varied significantly, from close to the freezing point of the solution to high temperature just short of causing any significant degradation of the active drug. It should be noted that the depression of freezing point and the elevation of boiling point in an electrolyte solution could be substantial and taken into account in deciding the range of temperature studied. Generally, a temperature range of 5-50° C. should suffice. However, if a change in the temperature of the dissolution media does not produce a statistically significant shift in the dissolution profile, then this stressor is not suitable to discern the differences between the test and the reference product. This may happen in the case of highly soluble drugs, in which case dissolution media should be selected to moderate the dissolution rates.

Dissolution profiles obtained at different temperatures reflect the inter-particulate and intermolecular interactions within the drug product and form an excellent basis of matching the thermodynamic potential of the drug products.

A significant iteration of this exercise can be a programmed heating of the dissolution solution allowing a single profile starting with a low temperature and then reaching to the highest limit selected. A statistically superimposable profile between drug products should indicate high level of inter-particle binding and interactions of drug product with the excipients.

To avoid any limitations due to solubility considerations, the current dissolution testing is conducted under sink conditions. However, in establishing a thermodynamic equivalence, this may not be necessary to maintain. Again, the purpose of thermodynamic equivalence demonstration is a comparison of the test product with a reference product and whatever testing condition that allows it without degrading the product is acceptable.

### Dielectric Properties

Drug solubility plays a significant role in the BCS for eligibility for waivers from bioequivalence testing. The changes in dielectric constant of the medium have a dominant effect on the solubility of the ionizable solute in which a higher dielectric constant can cause more ionization of the solute and results in more solubilization. As an example, water (DW, 298=78.5) has higher dissociation strength on ions in comparison with ethanol (DE, 298=24.2) which results in more solubilization power of ions in water (Table 3).

Given below is a theoretical model for solubility correlation in two different media or phases as following equation:

Log(S1/S2)=(0.4343×e²/2rkT)(1/D2)−(I/D1)
where S1 and S2 are the solubilities of the solute in media 1 and 2; e is the charge of an electron; r is the effective radius of the ion in the medium; k is the Boltzmann constant; T is the absolute temperature; and D1 and D2 are the dielectric constants of the media 1 and 2, respectively. Unfortunately, by using this equation, the predicted solubility values (when r values are known) or predicted r values (when solubility values are known) based on experimental data do not seem to be meaningful.

However, one can consider the constant value of (0.4343×e²/2rk) as AT for a specific solute and obtain:

Log(S1/S2)=(AT/T)(1/D2)−(I/D1)
where AT is a slope which can be calculated using two experimental solubility data points (e.g., solubility values in water and ethanol). The resulted AT values show indirect relation with temperature. This is expected, as it has indirect correlation with r, which has direct correlation with temperature. Also, it seems that AT values are not mainly affected by the structure of the solutes under study.

The main advantage of the proposed model is that it does not require any experimental solubility data in mixed solvents. Just two experimental solubility data points in mono-solvents and dielectric constants of solvent systems under consideration are employed in the prediction process. It almost provides good results, which might show its applicability in solubility prediction.

The main disadvantage of the proposed prediction method is that it is applicable only for the solubility prediction of electrolytes or zwitterions in which the ionization is the dominant parameter and the phenomenon could be represented.

The rate of release of drug products into solution is determined by the kinetic energy as studied above in the variation of the temperature but it also affected by the dielectric properties of the dissolution medium; the dielectric constant also affects the solubility and thus the driving force for the release of drug molecules from the drug products. Given below is a listing of the dielectric constants of various media that may be suitable for this testing. One reason why these have not been tested is because the focus of most of the research in the past has been to emulate a physiological model rather than examine the drug product under conditions to yield differentiating dissolution rates.

TABLE 3
Dielectric Constant of Various Liquids

| Compound | Dielectric Constant |
|---|---|
| Vacuum | 1.0 |
| Pyridine | 1.1 |
| Butane | 1.4 |
| Methane | 1.7 |
| Pentane | 1.8 |
| Heptane | 1.9 |
| Chlorine | 2.0 |
| Decane | 2.0 |
| Dodecane | 2.0 |
| Hexane | 2.0 |
| Octane | 2.0 |
| Turpentine (wood) | 2.2 |
| Carbon tetrachloride | 2.2 |
| Toluene | 2.3 |
| Benzene | 2.3 |
| Palmitic Acid | 2.3 |
| Stearic Acid | 2.3 |
| Cumene | 2.4 |
| Styrene | 2.4 |
| Napthalene | 2.5 |
| Caproic acid | 2.6 |
| Carbon disulfide | 2.6 |
| Linoleic Acid | 2.7 |
| Pinene | 2.7 |
| Terpinene | 2.7 |
| Furan | 3.0 |
| Bromine | 3.1 |
| Cotton seed oil | 3.1 |
| Olive Oil | 3.1 |
| Resorcinol | 3.2 |
| Linseed Oil | 3.3 |
| Ether | 4.3 |
| Phenol | 4.3 |
| Castor Oil | 4.7 |
| Chloroform | 4.8 |
| Acetic Acid | 6.2 |
| Ethylamine | 6.3 |
| Aniline | 7.3 |
| Cresol | 10.6 |
| Propylene | 11.9 |
| Hexanol | 13.3 |
| Ammonia (aqua) | 16.5 |
| Acetone | 20.7 |
| Alcohol, propyl | 21.8 |
| Ethanol | 24.3 |
| Methanol | 33.1 |
| Ethylene glycol | 37.0 |
| Furfural | 42.0 |
| Glycerol | 42.5 |
| Hydrazine | 52.0 |
| Glycerin | 55.0 |
| Water | 80.4 |

### Surfactants

Surfactants alter the solubility of drug by micellization; enhanced wettability of the surface of the drug product can additionally affect the dissolution rates by reducing the coating of particles by air and also by affecting the energy of inter-particulate and inter-molecular bonds. If the addition of a surfactant changes the dissolution profile and the test and reference product show similar changes, it will indicate equality of the chemical potential at the site of administration. The choice of surfactants can include both polar or non-polar surfactants and a combination of surfactants.

pH

The effect of dissolution medium pH on dissolution of drugs has been well studied and the current biowaivers guidelines include demonstrating dissolution at various pH as a pre-requisite to requesting waivers from bioequivalence testing. The premise behind the current requirements is to correlate these profiles in a physiological milieu. Given that the excipients as well as the active drug is highly affected in its state of ionization based on pH, the real test of similarity will involve studying dissolution throughout the available pH range, perhaps from 1 to 14 and then comparing the drug products based on the superimposability of these profiles. Subtle differences in the physicochemical characteristics would readily become evident under these conditions.

### Osmolality

Whereas buffers are used to adjust the pH for dissolution testing, the buffer capacity and thus the osmolality of the dissolution medium has not been the focus of any significant studies. The osmolality plays a significant role in determining the electrostatic interactions between the drug product, the released molecules and the components of the dissolution medium. The range of osmolality should be wide, likely between 100 mOsm/L to 1000 mOsm/L and even higher to cause a significant change in the dissolution profile of the reference test product. There is no need to select an osmolality based on any physiologic relevance as it is used merely as a thermodynamic stressor. If suitable osmolality to induce changes in the dissolution profile are not observed then other more suitable thermodynamic stressor should be chosen.

### Lipophilicity

While the stress of dielectric characteristics determines the polarity of the medium, the lipophilicity of the dissolution medium can play a significant role of thermodynamic stressor. In many instances, selecting appropriate lipophilicity of the dissolution medium can substantially slow down or enhance the dissolution rates. The goal of including this stressor is to provide a highly valuable thermodynamic stressor to create a matrix of dissolution profiles.

### Bipolarity

A biphasic system with both polar and non-polar characteristics can provide an excellent thermodynamic stressor when other stressors may not be so effective. Ideally, an emulsion (o/w type) dissolution medium should be used to determine if this constitutes a thermodynamic stressor. Alternately a w/o type dissolution medium can also be used. An interesting biphasic system can be the milk containing various percentages of fats; other biphasic systems such as coacervates etc., can also be very useful.

### Electrical Field

An electrical field applied to the dissolution medium can induce significant movement of charged species, both that come form the drug product and those that are present in the dissolution medium. Voltage ranges from 1-20 DC volt can be very useful in creating an electrical field that can modify the release characteristics of the drug products.

### Physical Stress

The normal physical stress provided in a typical dissolution apparatus is a paddle moving the liquid as seen in the most widely used USP apparatus. A variety of other mixing means may have variable effect on the release profile of the active drug. The purpose of choose alternates is not to simulate any physiological conditions but to provide an environment where the dissolution profiles are shifted and then observing the differences between the test and the reference drug products. The physical stress may involve using apparatus that allow rolling of the drug product, mechanical vibrations, ultrasonic vibrations, orbital motion, linear motion vertically or horizontally and any other type that can result in a change in the dissolution profile.

### Sink Condition

Most dissolution testing is conducted under sink conditions to obviate the effect of drug saturation solubility on the dissolution rates; however, in reality, the site of administration may not have sufficient volume and thus not emulate a sink condition. The dissolution testing should be conducted at both sink and non-sink condition making the solubility parameter as another thermodynamic stress. In those cases where the drug is readily dissolved, dissolution media should chosen to reduce the dissolution rates to allow reasonable ranges of comparisons. Under sink conditions, the dissolution profile will change but not the plateau level; under non-sunk conditions, both can change providing better opportunities of comparing two drug products.

### Duration of Testing

Since the solubility of the active drug is constant under specific conditions, every dissolution test will eventually lead to a plateau effect as it is entirely driven by the solubility. How fast a dissolution profile reaches the plateau is determined by the structural differences in the drug products. An appropriate test method will avoid reaching a plateau and generally short-term testing will reveal greater information about the differences between drug products; studies conducted for less than 60 minutes will be most relevant.

### Proposed Protocols

TEST™ for Bioequivalence will require establishing a matrix of tests, using at least two independent thermodynamic stressors independent of each other such as temperature and polarity. The next step will be to identify at least three conditions for each stressor that demonstrate a clear shift in the dissolution profile and then generating a dissolution matrix; for example, in the case of two stressors and three conditions, it will yield a matrix of nine test profiles. Applying the same conditions of testing to both the reference and test drug products, a statistical analysis of the reproducibility of effects between the test and reference drug product would establish thermodynamic equivalence. An almost unlimited set of conditions can be applied to force a variation at three levels for every drug.

### SUMMARY

In summary, there are several concrete reasons for challenging the current methodology of testing bioequivalence.
The purpose of bioequivalence testing should be to identify any differences in two drug products, regardless of their projected impact on clinical efficacy. This is necessary since the correlation between formulation differences and clinical efficacy will never be fully understood.
The pharmacokinetic profile of a drug product does not constitute a good surrogate for the concentration of drug at the site of action; this is mostly presumptuous.
The ultimate test of bioequivalence should be the rate and extent of delivery of drug at the site of administration, not the site of action, as currently required; this is a significant step to consider since the site of action for most drugs remains elusive and almost never accessible for sampling.
The potential for a drug to be absorbed in the biological system is proportional to the chemical potential of the drug at the site of administration, which is in turn proportional to the thermodynamic potential of the drug product.
The thermodynamic potential of drug products can be measured by subjecting them to conditions wherein the dissolution profiles are altered; observing similar changes between the test and the reference drug product should establish thermodynamic equivalence.
The thermodynamic equivalence can be applied to every type of dosage form regardless of its release characteristics since the test establishes that under all conditions the chemical potential will be identical to the innovator product or a reference product.
The thermodynamic potential test can be used as a routine test to monitor the quality of the product throughout the lifetime of the product.
There is no rationale for exposing healthy humans to drug testing when the results show that these data are inconclusive.
Significant cost savings and reduction in the development time can be achieved by eliminating all in vivo testing.
The use of thermodynamic equivalence will eliminate the exposure to humans, reduce the cost of development and allow faster regulatory approvals—all needed to bring the cost of drugs on a global basis.

What is claimed is:

1. A method for measuring the thermodynamic potential of the release of an active drug substance contained in a drug product comprising comparing a test drug product with a reference drug product by a set of statistically differentiated dissolution profiles of the active drug substance contained in the drug products.

2. A method for determining drug bioequivalence comprising measuring the thermodynamic potential between a test drug substance and a reference drug substance under conditions that yield a difference in their dissolution profiles, wherein equivalence of thermodynamic potential demonstrates drug bioequivalence.

3. The method of claim 2, wherein a matrix of dissolution data and comparability of said data between the test drug substance and the reference drug substance demonstrate drug bioequivalence.

4. A method for determining drug bioequivalence comprising measuring the thermodynamic potential between a test drug substance and a reference drug substance under conditions wherein the inter-particulate binding and the intermolecular interactions are disrupted.

5. A method for determining drug bioequivalence comprising measuring the thermodynamic potential between a test drug substance and a reference drug substance under thermodynamic stress conditions that modify the release characteristics of the test and reference drug substances to yield a difference in their dissolution profiles, wherein the stress condition is chosen based on its ability to alter the dissolution profile allowing the determination of drug equivalence.

6. The method of claim 5, wherein the stress condition is chosen from temperature, pH, dielectric constant, polarity (and bipolarity), osmolality, electrical field and various permutations and combinations of these conditions.

7. The method of claim 3, wherein at least three conditions for each stressor that demonstrate a clear shift in the dissolution profile to generate the dissolution matrix applying the same conditions of testing to both the reference and test drug substances, and wherein a statistical analysis of the reproducibility of effects between the test and reference drug product establish drug bioequivalence.

8. The method of claim 7, wherein two stressors and three conditions are applied to both the reference and test drug substances to yield a matrix of nine test profiles.

9. The method of claim 1, wherein the set of dissolution profiles is obtained in a variety of dissolution media.

10. The method of claim 9, wherein the dissolution medium has a pH in the range of 1-14.

11. The method of claim 9, wherein the dissolution medium has an osmolality in a range of 100 to 1000 mOsm/L.

12. The method of claim 9, wherein the dissolution medium has a dielectric constant value of more than 1 and less than 81.

13. The method of claim 9, wherein the dissolution medium further comprise a non-ionic surfactant, an ionic surfactant or a combination thereof.

14. The method of claim 13, wherein the concentration of each of the surfactant is below its critical micelle concentration.

15. The method of claim 9, wherein the dissolution medium comprises a mixture of a polar liquid, a non-polar liquid or a plurality of polar and non-polar liquids.

16. The method of claim 9, wherein the chemical composition of the dissolution medium is an oil-in-water or water-in-oil emulsion.

17. The method of claim 9, wherein the chemical composition of the dissolution medium is a coacervate.

18. The method of claim 9, wherein the chemical composition of the dissolution medium is milk.

19. The method of claim 9, wherein the dissolution media are subjected to a variety of physical conditions.

20. The method of claim 19, wherein the physical condition is temperature within a range of 5 to 50° C.

21. The method of claim 19, wherein the physical condition is an electrical field applied to the dissolution medium from 1-50 volt direct current.

22. The method of claim 19, wherein the physical condition is mixing of the dissolution by a stirrer, paddle, mechanical vibrator, or ultrasonic vibrator placed inside the dissolution medium.

23. The method of claim 19, wherein the physical condition is the rocking, shaking, rotating and vibrating the dissolution medium.

24. The method of claim 1, wherein the dissolution profiles is created for less than one hour.

25. The method of claim 1, wherein the dissolution profile is created under sink condition.

26. The method of claim 1, wherein the dissolution profile is created under non-sink condition.

27. The method of claim 1, wherein the drug product is a solid, semi-solid, liquid, powder or a gel.

28. The method of claim 1, wherein the method is used for establishing bioequivalence between a reference and a test drug product by comparing their dissolution profiles declaring bioequivalence when the dissolution profiles are statistically insignificant between the test and the reference product.

29. The method of claim 28, wherein the test product is a generic product and the reference product is the innovator product for said generic.

30. The method of claim 28, wherein the bioequivalence is established as a part of regulatory approval of a generic product.

31. The method of claim 28, wherein the bioequivalence is established as a part of complying with regulatory requirements of SUPAC.

32. The method of claim 1, wherein the characterization is used to release the drug product in the normal course of manufacturing.
